(19) United States
(12) Patent Application Publication
(10) Pub. No.: US 2007/0039835 A1
(43) Pub. Date: Feb. 22, 2007

(54) Microfluidic flow monitoring device

(75) Inventors: Joel Stephane Rossier, Vionnaz (CH); Patrick Morier, Bionay (CH); Frederic Reymond, La Conversion (CH)

Correspondence Address: HOWSON AND HOWSON, SUITE 210, 501 OFFICE CENTER DRIVE, FT WASHINGTON, PA, 19034, US

(73) Assignee: Diagno Swiss S.A., Monthey (CH)

(21) Appl. No.: 10/571,986

(22) PCT Filed: Sep. 15, 2004
(86) PCT No.: PCT/EP04/10733

§ 371 Date: Mar. 14, 2006

Related U.S. Application Data
(60) Provisional application No. 60/503,616, filed on Sep. 15, 2003.

(51) Int. Cl. G01F 1/64; G01N 33/487

(52) U.S. Cl. 205/792; 204/403.01

(57) ABSTRACT

An electrochemical flow monitoring device comprises a microfluidic system comprising at least one covered microchannel (3) having an inlet (4) and an outlet (5). A pressure difference is applied between the inlet and the outlet of the microfluidic system, for example by changing the relative heights of the inlet (4) and outlet (5), such as to generate a flow of solution within the microchannel (3). The microfluidic system has at least one electrode (8) for monitoring said flow of solution by measuring an electrochemical property of said solution.

### BACKGROUND TO THE INVENTION

This invention relates to a microfluidic flow monitoring device and to a method of performing an analytical assay.

Development of microfluidic devices for manipulating fluids has been a center of interest in research for more than 10 years. The use of microfluidics has become popular because it enables the analysis of minute quantities of fluid sample by means such as capillary electrophoresis or nanoelectrospray mass spectrometry. The applications of these “fluidic microchips” or microfluidic devices are numerous and reactions such as PCRs (polymerase chain reactions), hybridisations, immunoassays, syntheses etc have been developed on these microsystems.

One constant preoccupation is to find a better way to induce and control the desired flow profile, flow direction and flow rate during the analyses. Some microfluidic systems work with interconnected covered microchannels which do not need valves to direct fluids in the right direction. An understanding of the high voltage distribution is sufficient to enable the right flow profile and direction during capillary electrophoresis, for instance. However, these systems necessitate a perfect control of the wall surface during the analyses, which is difficult in real sample handling. Other systems work with integrated valves and pumps in order to distribute the solution at the correct flow rate and in the correct direction. Such systems need either to integrate microvalves in the device itself or to be connected to external valves and pumps by means of capillary tubing. These approaches can be cumbersome when integrated into disposable microfluidic systems, notably because they increase the cost of the final sensor device. In addition, the connection of a disposable part to an external capillary may become difficult to achieve without any contamination or dead volume.

To overcome these problems, pumpless systems have been proposed that provide fluidics by different means such as capillary filling, centrifugal force (hydrophobic gate Gyros, Gamera), aspiration by wiping (WO 01/26813, Caliper) or by using gravity to apply a pressure difference in order to generate a flow (WO03/008102, WO00/53320, WO01/26813).

The use of such pressure to generate a flow has seldom been applied to microfluidics because of the difficulty of efficiently monitoring the flow rate inside the chip while applying the pressure difference between the two covered microchannel ends. Indeed, the volume present in the microchannel is so small that it is difficult not only to monitor the flow rate but even to be sure that the sample has entered the channel.

### SUMMARY OF THE INVENTION

It is an aim of this invention to enable the in-situ measurement of the flow induced when using a microfluidic device as an analytical or reaction tool, and to measure this flow by means of an electrochemical event. The systematic measurement of this flow may serve to finally correct the result of the analysis performed in the chip

The present invention relies on the fact that a change occurring as a result of pressure on a fluid or a fluid flow inside a covered microchannel may be measured by electrochemical means. It is thus another aim of the present invention to provide an apparatus and method that allows the measurement of an electrochemical signal indicating such a change or flow in a microfluidic device and to generate a fluid flow by application of a pressure difference between the inlet and the outlet of a microchannel.

The present invention provides an electrochemical flow monitoring device, comprising:

a microfluidic system comprising at least one covered microchannel having an inlet and an outlet;

means for applying a pressure difference between the inlet and the outlet of said microfluidic system such as to generate a flow of solution within said covered microchannel;

wherein the microfluidic system has at least one electrode for monitoring said flow of solution by measuring an electrochemical property of said solution.

The solution may comprise a reporter molecule for monitoring said flow of solution by measuring said electrochemical property of said solution.

In one embodiment, the pressure difference is induced by gravity, namely by a difference in solution height between the inlet and the outlet of said covered microchannel. Alternatively, said means for applying a pressure difference may comprise an external actuator. A pumping system may for example be put in contact with the inlet of the microchannel and actuated in order to impose a pressure on the fluid present at this inlet and/or within the microchannel, thereby generating a solution flow within said microchip. Alternatively or additionally, an underpressure may be generated at the outlet of the microchannel in order to generate aspiration of a fluid through the microchannel.

In another embodiment, the pressure difference is generated by imposing an acceleration to the microfluidic system. In some cases, this pressure difference induced by acceleration may be superimposed to the pressure difference induced by gravity or to that applied by way of an external actuator. Otherwise, this acceleration may be induced by the rapid displacement of the microfluidic system or of a solid support on or in which the microfluidic system is placed. In some embodiments, this displacement consists in a vertical lift of the microfluidic system or of its solid support, and this vertical lift may be achieved by means of a plug mechanism or with a spring placed under the microfluidic system or its support. With a vertical lift of 1 cm in 0.01 second, the induced acceleration is 5 g, namely five times the effect of the gravitation force. In another embodiment, the acceleration can be induced by rotating the microfluidic system or its support, thereby using centrifugal forces to apply a pressure difference between the inlet and the outlet of the microchannel. To this end, the inlet and the outlet of the microchannel should normally not be positioned at the same distance from the center of rotation, so as to create a different momentum at the inlet and at the outlet of the microchannel, thereby imposing a pressure difference between the two extremities of the microchannel. To achieve this, the microchannel could for example be positioned in such a manner that the line joining the inlet to the outlet exhibits an angle different from ninety degrees to the normal of the rotation axis.

The electrochemical property may be a specific conductivity or a reduction or oxidation (redox) property. The redox property may comprise the ability of a molecule e.g. ferrocene, ferrocene carboxylic acid, hexacyanoferrate or oxygen, dissolved in said solution, to be reduced or, respectively, oxidized.

The microfluidic system may comprise a material selected from polymer, glass, ceramic, another flow tied material and a combination thereof. The microfluidic system may comprise a multi-layer body. In some embodiments, the microfluidic system is fabricated by plasma etching and/or laser photoablation of a multi-layer body. These fabrication processes may indeed be advantageously used to manufacture microfluidic systems with one or several integrated electrode(s). Embossing, injection molding, UV-Liga, polymer casting, silicon etching and any other microfabrication technique may also be used to fabricate the microfluidic system. In some other embodiments, the microfluidic system may be made of or comprise a light-transparent material, thereby for example enabling optical detection of an analyte.

The microchannel is sealed, and it may be covered by one of a lamination, a sealing plate and a plate fixed over said microchannel and maintained by external pressure.

The microfluidic system may advantageously comprise a biological material such as but not limited to an enzyme, an antibody, an antigen, an oligonucleotide, a DNA, a DNA strain or a cell. In some embodiments, this biological material may be immobilized on the walls of the microchannel and/or on the electrode used to measure an electrochemical property within said microchannel. The flow monitoring device of this invention may then be directly used to perform an assay, during which the flow of solution through the microchannel can be monitored electrochemically and where the electrochemical flow measurement may even be used to correct for the final result of the assay.

The at least one electrode may be composed of a conductive surface such as a metal surface, carbon or a liquid/liquid interface.

The flow of solution can, for example, be used to perform incubation of a solution in an affinity sorbent assay.

The invention also provides a method of performing an analytical assay comprising the steps of:

(a) providing a flow monitoring device as defined above;

(b) depositing a solution at the inlet of said covered microchannel;

(c) applying a pressure difference between the inlet and outlet of said microchannel in order to generate a flow of said solution in said microchannel; and

(d) measuring an electrochemical property of said flowing solution, which property depends on the flow rate of said solution in said microchannel, by means of said at least one electrode of said microsystem.

In embodiments of the method, steps b) to d) are repeated in order to perform a multistep assay.

The method may comprise stopping the application of said pressure difference in order to detect an analyte present in said solution. For example, a liquid immiscible with said solution may be added to at least one of said inlet and/or said outlet. In another embodiment, the solution flow may be blocked by mechanical means, for example by obstructing the inlet and/or outlet of the microchannel. In a further embodiment, a bubble may be generated electrochemically in the microchannel, notably at the integrated electrode, so as to block the solution flow within the microchannel.

The surface tension at the inlet and/or outlet of the microchannel may also be adapted in order to prevent a solution to flow out of the microchannel. In another embodiment, the microchannel may first be filled by capillarity upon deposition of a solution at the inlet of the microchannel; once filled, a pressure difference may then be applied between the inlet and outlet of the microchannel so as to generate a flow of solution that is monitored by electrochemical means.

The measured flow rate may be used to correct the final result of an assay performed directly with the flow monitoring device of this invention. In this case, it may indeed be advantageous to prevent any flow of solution during the analyte detection. In some embodiments of this invention, a reporter molecule may be added to the sample and/or reagent solution(s) in order to monitor the fluid flow within the micro-channel. The analyte that has to be detected during the assay may for example be electroactive or highly conductive, so that its presence and/or its concentration may be directly determined by the flow monitoring device of this invention using an electrochemical property of this analyte. In some embodiment, more than one analytes may be assayed simultaneously in one single microchannel.

### BRIEF DESCRIPTION OF THE DRAWINGS

In order that the invention may be more readily understood, particular embodiments thereof will now be described, byway of example only, with reference to the accompanying drawings, in which:

FIG. 1 is a schematic sectional view of a microfluidic device according to an embodiment of the invention;

FIG. 2 is a schematic sectional view of a microfluidic device according to an alternative embodiment;

FIG. 3 is a schematic plan view of the device shown in FIG. 2;

FIG. 4 is a graph of redox current against time for the purpose of monitoring flow using the device of FIG. 1;

FIG. 5 shows the current of FIG. 4 plotted against flow rate;

FIG. 6 shows the device of FIG. 1 in a tilted condition;

FIG. 7 is a graph of redox current against time for the purpose of monitoring flow using the device as shown in FIG. 6;

FIG. 8 shows the current of FIG. 7 plotted against height difference between the two reservoirs of the device;

FIG. 9 is a graph of flow rate against height difference; and

FIG. 10 is a graph showing the amperometric detection of ferrocene carboxylic acid.

### DETAILED DESCRIPTION OF PARTICULAR EMBODIMENTS

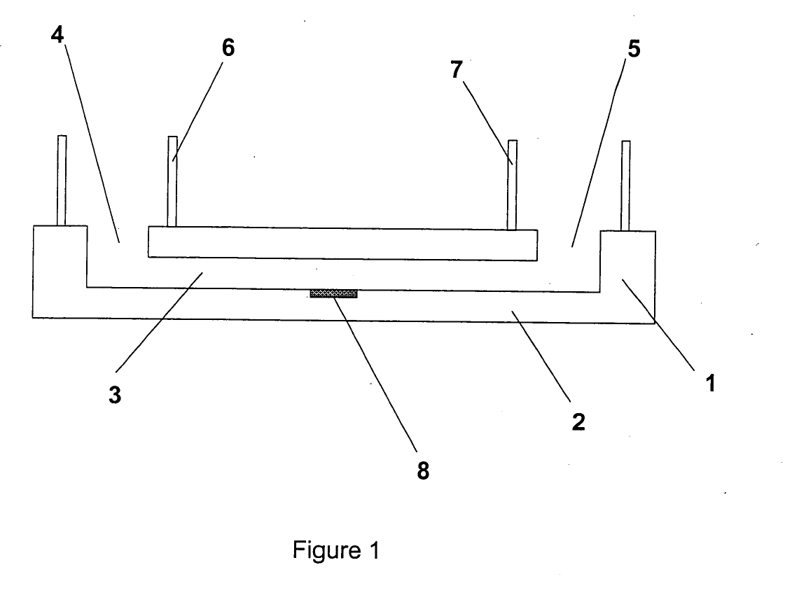

FIG. 1 shows a microfluidic device 1 (also referred to hereinafter as a microchip). Whilst a polymer-based microfluidic device is preferred, different devices, including glass, silicon, ceramic materials, etc can also be used.

The microchip 1 is composed of a body 2, said body comprising a covered microchannel 3 having a least one dimension compatible with laminar flow conditions. The covered microchannel has at least one inlet 4 and one outlet 5, the inlet and outlet each being composed of a hole, a tip or a venting material enabling the passage of fluids (gas or liquid). In this example, the inlet 4 and the outlet 5 are respectively surrounded by an inlet reservoir 6 and an outlet reservoir 7. A detector 8, comprising an integrated electrode, is in contact with the body 2 such as to enable the detection of changes due to the presence and/or the flow rate change of a fluid in the covered microchannel 3.

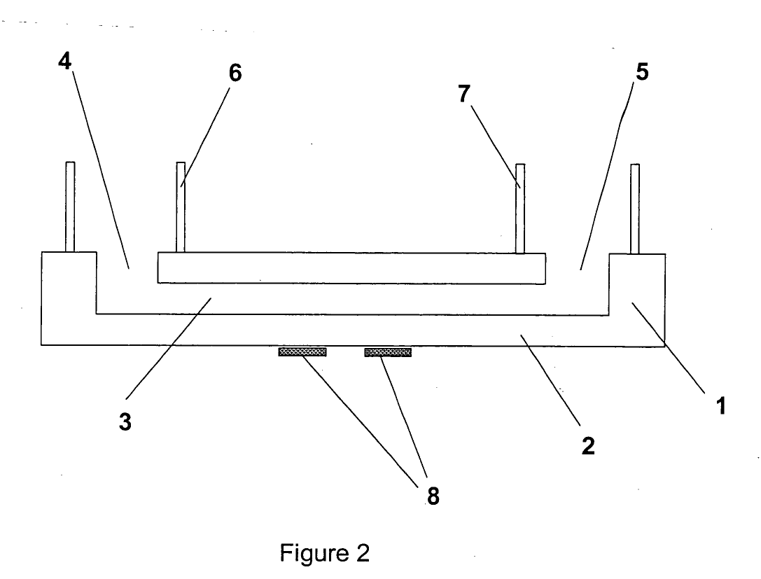

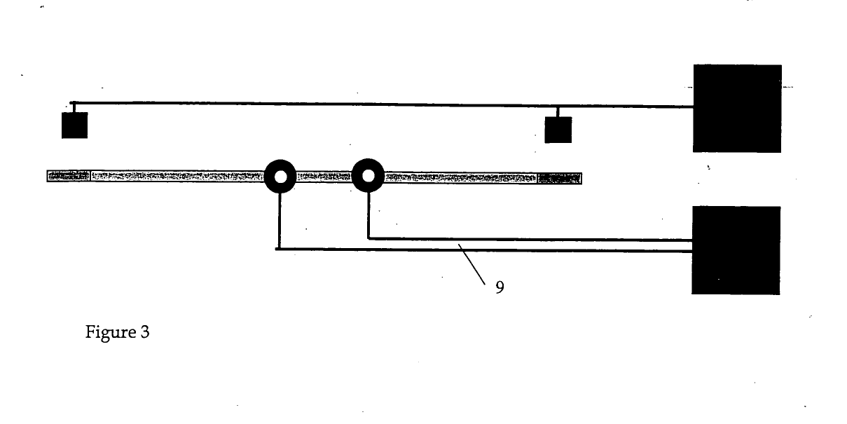

FIG. 2 shows a device similar to that shown in FIG. 2, but comprising contactless electrodes 8 instead of the integrated electrode. As shown in FIG. 3, the electrodes 8 are in contact with conductive tracks 9 patterned on the substrate of the device, the conductive tracks enabling electrical connection to an external interface (not shown) for electrochemical measurements.

The interface connects the microfluidic device to a detection system and/or to a pumping system. The interface comprises a fluidic connection insuring a good sealing between the pumping system and the covered microchannel as well as an electric connection, between the detector through the conductive tracks 9 and the electronic detection system.

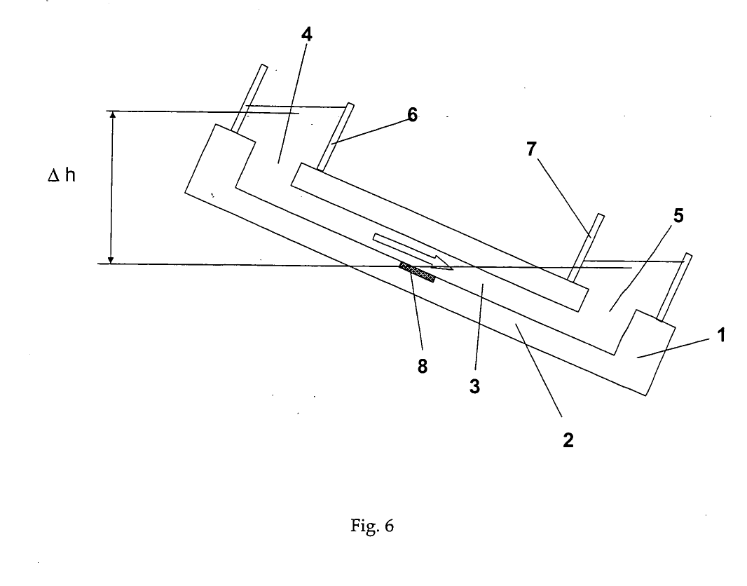

The pumping system is a system which is able to generate a pressure difference between the inlet and the outlet of a microchannel. Two different pumping systems have been used in the present invention: (i) a syringe pump (Kd Scientific, model 200, equipped with Hamilton syringes, 100 μL—not shown) and (ii) a tiltable plate, discussed below with reference to FIG. 6.

The electronic detection system (not shown) comprises any system adapted to perform an electrochemical measurement, e.g. a potentiostat, an impedance apparatus, etc. In the embodiments of the invention described herein, the electronic detector comprises a multiplexer part, which allows the measurement of several microchannel simultaneously, and a potentiostat which is able to apply a potential and to measure a current (here, the potentiostat and the multiplexer part are from Palm Instruments BV, Netherlands).

The microfluidic device 1 is provided by a plasma etched chip fabricated with a technology fully described elsewhere (Rossier et al. Plasma etched polymer microelectrochemical systems; Lab Chip, 2002, 2, 145-150). The geometry of the microfluidic device is presented in Table 1 where the principal parameters of said microfluidic device are listed.

TABLE 1
parameter list of the covered microchannels used in the present invention

| | | | | Eq 6:Poiseuille | |
|---|---|---|---|---|---|
| | Reality | Eq 1:Bartlett | Eq 4:Levich simulation | simulation | Eq 7 |
| Channel radius | — | — | — | 55 μm | 55 μm |
| Channel height | 67 μm | — | 67 μm | — | 67 μm |
| Channel width | 160 μm | — | 150 μm | — | 140 μm |
| Surface/Volume ratio | 46500 | | | 77000 | |
| Covered micro- channel shape | 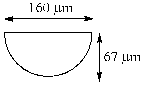 | | 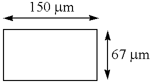 | 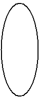 | |
| Electrode radius | 26 μm | 26 μm | — | — | |
| Electrode band width | — | | 100 μm | — | 100 μm |
| Electrode band | — | | 30 μm | — | 30 μm |
| length | | | | | |
| Electrode area | 2.1 nm² | 2.1 nm² | 2.1 nm² | — | 2.1 nm² |
| Electrode recess | 8 μm | 8 μm | 0 | — | 8 μm |
| Electrode shape | 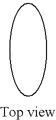 | 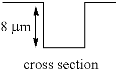 | 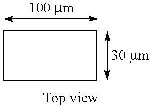 | | 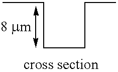 |

The use of similar microfluidic devices has already been demonstrated in connection with a pumping device wherein a pump delivered a constant flow rate in opposition to a constant pressure inside the channel. (Rossier et al. Plasma etched polymer microelectrochemical systems; Lab Chip, 2002, 2, 145-150). The flow rate dependence was studied with this syringe pump and it was shown that the electrical current detected had a direct relationship with the imposed flow rate.

In the case of a pressure driven system, the reality is sometimes much more complex because at the micrometer scale different phenomena also occur such as a change in the surface tension or bubble trapping, meaning that a given pressure is sometimes not enough to pump the same amount of liquid at the same rate in two similar microfluidic devices. If such a device is to work in a predictable way, it cannot be assumed that applying the same pressure will lead to the same events inside the covered microchannel. Therefore, according to the invention, the detectors 8 are used to probe for the presence or the replacement of fluids and/or to ensure that the flow rate induced by the constant pressure is correct. Electrochemical methods are also used to achieve such measurement. In this manner, the flow monitoring device of this invention may advantageously be part of a foolproof assay platform, i.e. an assay platform in which all the microfluidic steps are controlled by determination of the presence of a solution and/or by monitoring of the solution flow within the microchannel, thereby enabling for instance the production of a report concerning all the microfluidic events that occurred during the assay or to correct the final signal as a function of these microfluidic events.

Fluid Change in the Covered Microchannel Monitored by Conductivity

The contact or contactless electrodes 8 of FIG. 1 or 2 respectively can be used to probe for changes of fluid inside the microchip 1, for example, when air is changed to aqueous solution or when some resistive liquid (e.g. pure water) is followed by a sample of serum, plasma or blood (containing salt). A change in the measured conductivity can show that a sample, a washing solution or a reagent solution has correctly traveled through the microfluidic device 1. For example, the microchannel 3 is first filled with a resistive fluid, in this case air, and is then filled by capillary action with a salted aqueous solution (100 mM Phosphate, 100 mM KCl); then the salted aqueous solution is replaced with pure water. The change in conductivity can be measured and proves that the different fluids have traveled along the channel.

Syringe Pump Induced Flow Rate Monitoring by means of Electrochemistry

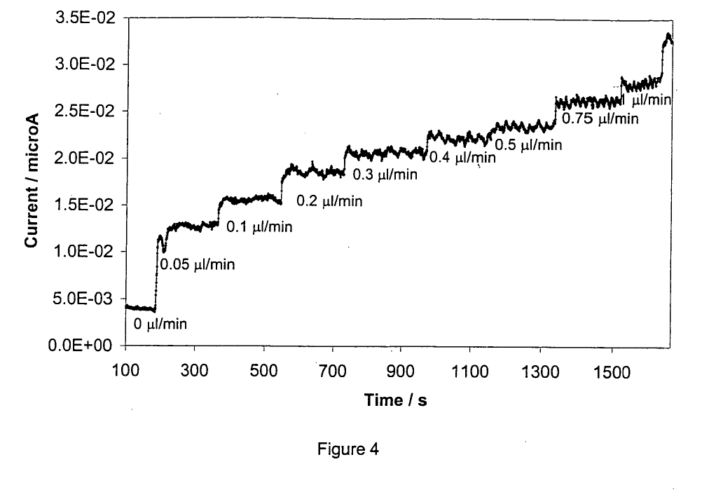

In an alternative use of the inventive device, the flow rate of a solution can be monitored by measurement of a redox marker added to the solution. To demonstrate the principle of this flow monitoring, an experiment has been conducted by connecting the microfluidic device 1 of FIG. 2 to a syringe pump, comprising of a 10 μL Hamilton syringe actuated with a Kd Scientific pump, by means of an interface. The role of this interface is firstly to connect the microchannel 3 to a tube for fluidic connection but also to connect the two working electrodes 8 through the conducting tracks 9 shown in FIG. 3. The microchannel 3 is then filled with a solution of a redox active molecule (0.5 mM ferrocene carboxylic acid (FC) in 100 mM phosphate buffer and 30 mM KCl). The solution is then aspirated at different flow rates between 0 and 1.5 μLmin⁻¹ imposed by the syringe pump. The electrical current is continuously monitored and the convection flow rate is regularly increased inducing a measurable change in electrical signal. FIG. 4 shows the current plotted against time. At a flow rate of zero, the current value of approx 3.8 nA represents uniquely diffusional flow towards the electrode. The theoretical current is given by the following expression developed by Bartlett for recessed microelectrodes (Bartlett, P.N., J. Electroanal. Chem., 1998, 453, 49-60.)
I=f(τ)4nFDcr Eq 1

Where
I is the steady state current,
n the number of exchanged electrons per molecule,
F the Faraday constant,
D the diffusion coefficient (here, D=5.7×10⁻¹⁰ m²s⁻¹ for FC)
r the radius of the disc electrode (here 26 μm)
c the concentration of the redox molecule
   and where
   f(t)=B′t^{-1/2}+C′ exp(2D′t^{-1/2}) Eq 2
   with
   t=Dt/r² Eq 3
   with t=here for the steady state current after 30 s. Finally B′, C′, and D′ are constants presented in (Bartlett, P.N., J. Electroanal. Chem., 1998, 453, 49-60, Table 5) for different dr/r values (dr being the depth of the recess)—which value here is approximately 0.3 (8 μm/26 μm), such that B′, C′ and D′ are respectively 0.4428, 0.5246 and 0.9926. With this theoretical expression, each working electrode should give a pure diffusional current of 2.05 nA, so the total current here for two electrodes should be 4.1 nA. This level of current is in relatively good agreement with the current actually measured at a flow rate of zero.

When the flow rate is enhanced by the syringe pump, an increase in current occurs revealing the renewal of the diffusion layer with fresh solution. The shape of the increase defines a step with a stable plateau after the steep current increase. This steepness shows that the inertia in the flow rate change is very low and that the electrochemical monitoring is immediate. It should also be mentioned that a frequency is measured in the plateau value, probably due to small steps induced by the syringe pump. In fact, the faster the flow rate, the higher the frequency, whereas the mean plateau value current remains stable. This observation shows again that it is possible to monitor slight changes in flow rate with this method.

The current of a band microelectrode inserted inside the microchannel, is dependent on the flow rate following the Levich equation:
I=0.925nFcL(l_{B}D)^{2/3}(Q/h_{M}²d)^{1/3} Eq 4

where

I is the amperometric current (A)

n is the number of electrons

F is the Faraday constant (96500 C mol⁻¹)

c is the analyte concentration (mol m⁻³)

L is the length of the electrode band (m)

l_{B} is the width of the electrode band (m)

D is the diffusion coefficient of the redox molecule (m² s⁻¹)

Q is the flow (m³ S⁻¹)

h_{M} and d are the height and the width of the channel respectively (m)

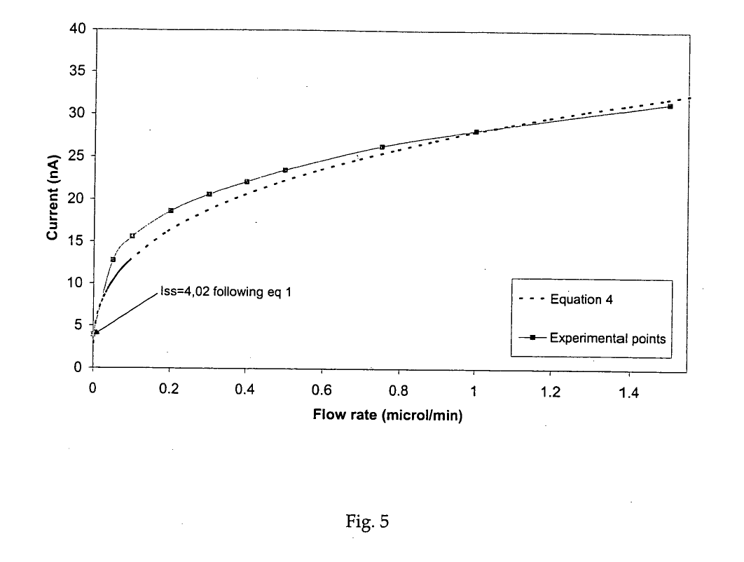

The evolution of the mean plateau current versus the flow rate is shown in FIG. 5. The evolution of the measured signal is dose to the Levich expression (Eq 4) as can be compared from the graph. Note that the deviation between the experimental and theoretical expression is due to the difference in geometry between the experimental microfluidic shape and the theroretical one. Indeed the Levich equation is applicable to a channel with an inlaid microband electrode in a microchannel whereas in the experiment we have a recessed disk microelectrode as shown in Table 1.

This experiment shows that the flow rate of a solution inside a channel can be quantitatively measured and that the measured current is in good agreement with the Levich model. These properties will be used in the following experiment where the flow rate is induced by the difference of pressure at the inlet and outlet of the microchannel.

Gravity Induced Flow Monitored by Electrochemical Means

Replacing the syringe pump with a pumpless system is possible using gravity. This fundamental approach, first solved by Blaise Pascal in the 17^{th} century, states that placing a fluid in a tube generates a pressure difference directly proportional to the elevation between the two tube ends. This law can be expressed by equation 5:
ΔP=ρgΔh Eq 5
where

ΔP is the pressure difference (Pa)

ρis the density (kg m⁻³)

g is the acceleration due to gravity (m S⁻²)

Δh is the height difference between the tube ends (m)

The second fundamental equation to calculate the flow rate generated by the difference of pressure inside a capillary was developed in the early 18^{th} century and reduced in a mathematical expression in 1860 known as the Poiseuille equation:
Q=ΔPπR⁴/8ηl Eq 6
where

Q is the flow (m³ s⁻¹)

R is the tube or capillary radius (m)

ηis the fluid viscosity (pa.s)

l is the length of the tube or the capillary (m)

The relationship between pressure difference (ΔP) and flow (Q) is therefore well-known and is usually sought to be overcome in standard microfluidic devices. Even when capillary electrophoresis was invented the so-called hydrodynamic pressure induced flow was identified and mathematically understood as described in the paper of Grushka et al. (Grushka, E, Effect of hydrostatic flow on the efficiency in capillary electrophoresis, J. Chromatog. 559 (1991), 81-93.). This effect is also present in microchip systems which are simply capillary tubes etched in glass, polymer or ceramic materials but which do not fundamentally differ from capillaries. It has already been described by different authors in microfluidic applications, for example by Boer et al. (Boer, G., Studies of Hydrostatic pressure effects in electrokinetically driven microTAS, MicroTAS systems '98 conference proceeding, Ed. D. J. Harrison, A. Van den Berg; Kluwer Academic Publishers; Banff, 98, p 53.)

To demonstrate the applicability of a pressure driven flow the following experiment has been performed with the microfluidic device 1 placed horizontally on a plate that can be tilted at different angles to generate a height difference between the reservoirs 6, 7, as shown in FIG. 6. When tilted up, a difference in height between the inlet reservoir 6 and the outlet reservoir 7 induces a flow of solution from the microchannel inlet towards the outlet. When tilted down, a gravitational flow is generated in the opposite direction.

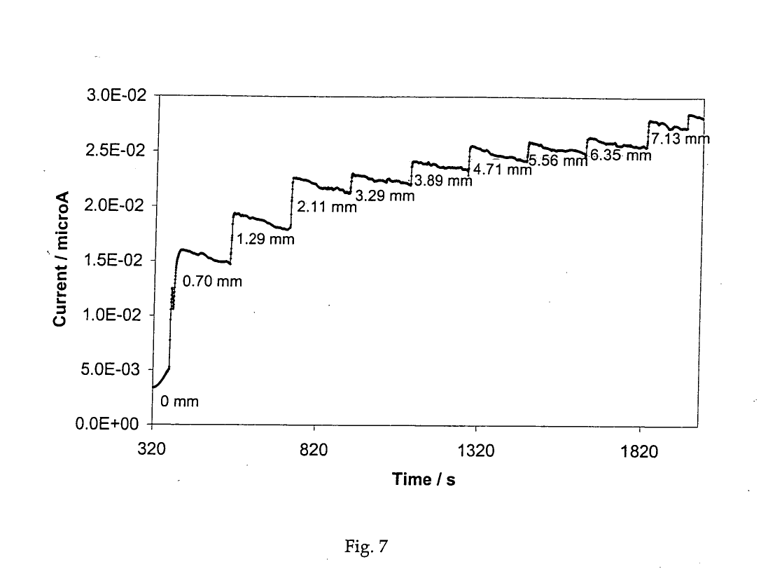

FIG. 7 shows the redox current obtained; the solution and microfluidic device used for the flow monitoring are the same as those used in FIG. 4 (0.5 mM of Ferrocene carboxylic acid in 100 mM phophate buffer and 10 mM KCl). The electrical current versus the difference in height shown in FIG. 7 presents a similar pattern to that of FIG. 4, in which the flow is induced by the syringe pump. At zero height difference the current is also measured in the range of 4 nA which corresponds to pure diffusional current. When a difference in height is introduced, the current increases rapidly and reaches a maximum, before showing a slight decrease. The increases are again sharp which shows the reactivity of the electrochemical measurement system. The slight decrease in the current signal indicates a slowdown in the flow rate because the height difference is compensated from one reservoir to the other when the solution is flowing through the covered microchannel.

Combining the Pascal, Poiseuille and Levich equations, it is possible to calculate the current as a function of the height difference between the inlet and the outlet of the covered microchannel:
I=0.925nFcL(l_{B}D)^{2/3}((ρgΔhπR⁴/8ηl)/h_{M}²d)^{1/3} Eq 7

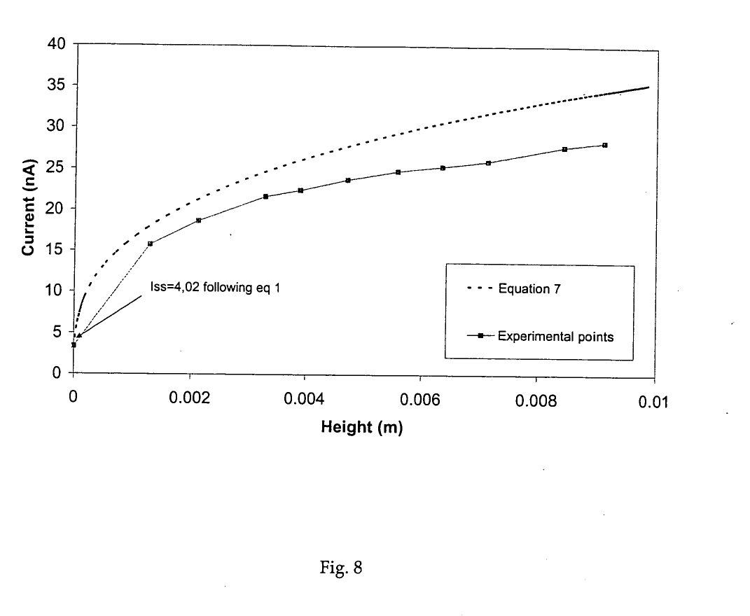

The evolution of the current as a function of the height difference is shown in FIG. 8, together with the analytical expression presented in Equation 7. The deviation observed between the analytical expression and the experimental results is mainly due to the fact that in Eq 7, the geometry given in valid for a tube with a circular section as presented in Table 1. In reality a pressure drop is induced because of the shape of the channel.

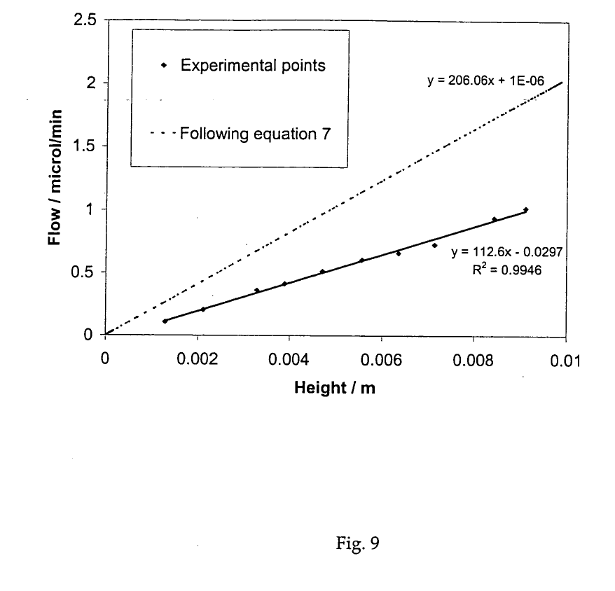

By using the calibration of current versus flow rate shown in FIG. 5 it is possible to know the actual flow rate induced by the difference in height, and this is plotted in FIG. 9. The real flow rate is lower than the analytical expression for the reason indicated above. Considering the difference in surface-to-volume ratio between the ideal capillary and the current chip it is easy to understand that the friction will be larger in the experiment, generating a pressure drop that reduces the flow rate.

For some applications, it may also be advantageous to use the plate supporting the microfluidic device 1 to stop the solution flow by placing it horizontally.

Blocking the Flow by Means of an Oil Plug in a Reservoir

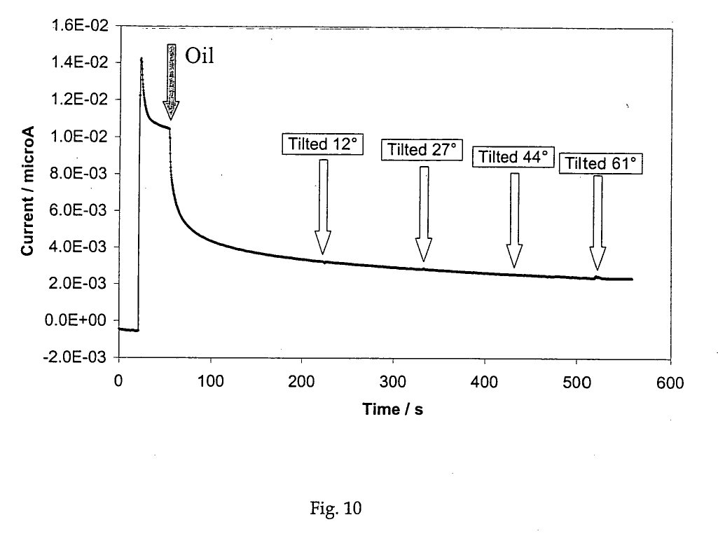

In microfluidic systems, a very small difference in solution height between the solution levels at the inlet and at the outlet extremities of the microchannel may induce a solution flow which can disturb the signal to be obtained with the sensor device. It is sometimes important to completely block the flow inside the channel in order to avoid siphoning that would continuously replace the solution in the channel with slow flow rate, even when the pressure difference is close to zero. In order to prevent this siphoning phenomenon, a drop of organic solution, immiscible with the solution present within the microchannel 3, can be added at the inlet 4 and/or outlet 5 of this microchannel so as to block the flow or prevent its generation. For example, an oil plug, such as a mineral or organic non miscible oil, can be added in the outlet reservoir 7 in order to make an interface with the water at the outlet. FIG. 10 shows the results of an experiment that demonstrates the efficient flow blockage of Ferrocene carboxylic acid solution when a mineral oil (Paraffin) is added to the waste reservoir instead of an aqueous solution. Indeed, before the addition of the oil, the current reaches about 10 nA, which represent about 0.03 ρLmin⁻¹ on the calibration of FIG. 5. When the oil is added, this slight flow is reduced and the current reaches about 4 nA which represents pure diffusional flow (Eq 1). To demonstrate the efficiency of this stop-flow-plug the microchip 1 is tilted at different angles which would have generated an increase of current (see FIG. 7) without the plug. It is remarkable here to see that even with an angle of 61° (Δh=8.7 mm) no current step is measured, revealing that no flow is induced inside the covered microchannel. This property of efficiently blocking the flow will be important during static incubation, for example during an enzymatic reaction, where the substrate is introduced and where an immobilized enzyme will generate a product with an increasing concentration. In some embodiments, this oil phase may serve as an ionode (i.e. an ion permeable membrane) for the detection or the referencing of the electrochemical event in the channel.

As an alternative, mechanical means may be used to dose the inlet and/or outlet of the microchannel so as to prevent any pressure difference between the two microchannel extremities and hence prevent any solution flow.

Integration of the Flow Monitoring Device in an Immunosorbent Assay Platform

In a preferred embodiment, the flow monitoring device of this invention is part of or consists in a platform for performing affinity assays such as but not limited to immunoassay, oligonucleotide hybridisation, protein interaction or drug discovery. In this case, an affinity partner (for example an antibody, flown at a concentration of 100 ρg/ml during 5 minutes followed by incubation of a blocking agent during 5 supplementary minutes, e.g. 2% bovine seum albumin) may be immobilised on the surface of the covered microchannel of the flow monitoring device of this invention. Then, the channel can be filled with a probe sample, for example a solution containing an analyte of interest such as an antigen (different concentrations from e.g. 0 to 10 uU/ml may be incubated under flow conditions during 5 minutes in a series of independent microchannel). In this case, the antigen and a conjugate antibody, which serves for the recognition of the antigen by specific binding and which is generally labelled with the enzyme such as e.g. alkaline phosphatase, are captured by the affinity partner immobilised on the microchannel surface; in order to monitor the flow by electrochemical means, the solution may contain a redox marker molecule such as ferrocene carboxylic acid (for example at a concentration of 0.25 mM); the microfluidic system may be a plasma etched polyimide chip sealed by lamination of a polyethylene/polyethylene terephthalate layer and comprising gold microelectrodes. In order to generate a solution flow, the microfluidic system is placed on a solid support which can be tilted with an angle adapted to generate the desired flow rate. Conductivity and/or amperometric detection can be performed in a continuous way such as to monitor the flow rate and detect any change due to modification of the angle, bubble formation or change in the viscosity of the solution for example.

Using the above-described device with eight microchannels in parallel and with ferrocene carboxylic acid as redox active reporter molecule, the current record for each channel can be plotted as a function of the tilting angle which is varied stepwise over time. It can then be observed that when the microchannels are horizontal (tilting of 0°), the current is very different in the different channels. The expected current for a pure diffusional steady-state (without convection is expected around 2.5 nA following equation 5). In each channel, the current is higher, probably revealing the presence of a slight flow. It may also be observed that the shape of the recorded current is also different in the various channels at low tilting degrees. However, when the tilting is greater (approximately 30°), the current shape is the same in the various microchannels. A jump in the current is measured upon increase of the tilting angle, and this current slightly decreases after the step. The mean amplitude of the current at the end of the experiment reaches about 6.5 nA (for 0.25 mM of ferrocene carboxylic acid (FC) in the antigen and conjugate serum solution). With the device of this invention, it may be deduced that the actual flow rate is approximately in the range of 0.1 ρL/min. This flow rate is slightly lower than the one reported in FIG. 7 for the same tilting when a pure aqueous solution (PC in PBS) was used for the characterisation of the tilting. The difference is probably due to different factors among them (1) the higher viscosity in the solution, which would slow down the flow according to Poiseuille equation (eq 10), (2) the decrease in the diffusion coefficient of FC in serum compared to simple buffer according to equation 5, and (3) a possible decrease of the specific surface area of the electrode after the protein coating steps.

This flow rate (approximately 0.1 μLmin⁻¹) is close to a rate enabling the total depletion of the probe sample, (according to eq 1, >80%) meaning that most of the molecules passing in the microchannel with a given diffusion coefficient should have the time to reach the wall surface and be captured by an affinity partner, which enables an efficient preconcentration of the analyte on the surface of the microchannel.

After this incubation step, which can require a few seconds to few hours, the solution in the inlet reservoir can be removed and replaced by a washing solution. This solution can either be less conductive than the probe sample solution and/or contain no redox molecule, thereby enabling to electrochemical differentiation between the sample and the washing solutions using the device of this invention and a definitive assessment that the washing solution has transited through the microchannels. The experiment shows that it is possible to fully monitor the fact that, after tilting the microfluidic system, the solution in the covered microchannel has changed since the current measured in the microchannel is close to 0 nA (no FC in the washing solution).

After this washing step, the sandwich affinity complex on the surface of the microchannels can be detected by depositing an enzymatic substrate solution, e.g. para-aminophenyl phosphate, at the inlets of the microchannels and by letting this substrate solution incubate. The substrate is then hydrolyzed into a product by the enzyme label on the conjugate and the product concentration increases with time. Just before starting the detection, it is advantageous to block the flow in the microchannels so as to ensure that the product will be accumulated in a constant volume with no siphoning effect. To achieve this goal an oil plug is added to the exit reservoir as presented above. The detection of the enzymatic product (e.g. para-aminophenol if para-aminophenyl phosphate is used as a substrate for the enzyme alkaline phosphatase) can then be performed in situ. The device of this invention, with its electrode(s) integrated in the microfluidic system, may advantageously be used for this purpose if the enzymatic product is to be detected by electrochemical means. Indeed, the product of the enzymatic reaction can for instance be detected by oxidation upon application of a potential at the integrated electrode(s). It is then possible by taking the measurements at different time intervals to follow the increase of the enzymatic product as a function of time and hence to determine the analyte concentration. A calibration has been achieved following the above procedure in which analyte solutions with antigen concentrations varying from 0 to 10 μU/ml were incubating in different microchannels.

It must be stated that the flow rate of each of the individual covered microchannels has been monitored with the device of this invention at each step of the entire assay, and that the measured signal can then serve as a recalibration in case one channel exhibits a larger flow rate than another.

By applying the above procedure, it has been demonstrated that the device of this invention allows not only to precisely monitor the flow of solution in a microfluidic system, but also to achieve high-performance multi-step assays such as immunological tests with no pumping system but with well-controlled flow rates.

The present invention enables direct flow monitoring within a covered microchannel and the use of an integrated electrochemical sensor to help understand the phenomena occurring in the microchannel.

All forms of the verb “to comprise” used in this specification have the meaning “to consist of or include”.

1. An electrochemical assay device with integrated amperometric flow monitoring means, characterised by:
a microfluidic system comprising at least one covered microchannel having an inlet and an outlet;
means for applying a pressure difference between the inlet and the outlet of said microfluidic system such as to generate a flow of solution within said covered microchannel; and
at least one electrode integrated in a wall portion of said microchannel, said electrode having precise size and location in said microchannel;
wherein said integrated electrode is adapted to monitor the solution flow at said integrated electrode by amperometric or conductivity measurement, and wherein said integrated electrode is in addition adapted to electrochemically detect an analyte of interest during an assay.

2. The device of claim 1, wherein said solution comprises a reporter molecule for monitoring the solution flow at said integrated electrode by amperometric measurement.

3-41. (canceled)

42. The device of claim 1, wherein said pressure difference is induced by gravity, namely by a difference in solution height between the inlet and the outlet of said covered microchannel.

43. The device of claim 42, wherein said microfluidic system is placed on or in a solid support which can be tilted in order to generate said difference in solution height between the inlet and the outlet of said covered microchannel.

44. The device of claim 42, wherein said microfluidic system is adapted to generate a flow of solution within said covered microchannel without any pumping means.

45. The device of claim 1, wherein said means for applying a pressure difference comprises an external actuator.

46. The device of claim 45, wherein said external actuator comprises means for imposing a pressure on the fluid present at the inlet and/or within said microchannel, thereby generating a solution flow within said microfluidic system.

47. The device of claim 45, wherein said external actuator comprises means for imposing an underpressure at the outlet of said microchannel, thereby enabling aspiration of said solution within said microchannel.

48. The device of claim 2, wherein said reporter molecule is any one of ferrocene, ferrocene carboxylic acid, hexacyanoferrate and oxygen.

49. The device of claim 1, wherein said microfluidic system comprises a material selected from polymer, glass, ceramic, another flow tied material and a combination thereof.

50. The device of claim 1, wherein said microfluidic system comprises a multi-layer body.

51. The device of claim 1, wherein said microfluidic system comprises a light-transparent material.

52. The device of claim 1, wherein said microfluidic system is fabricated by a process selected from plasma etching, laser photoablation, embossing, injection molding, UV-liga, polymer casting, silicon etching and any combination thereof.

53. The device of claim 1, wherein said microfluidic system comprises a network of microchannels.

54. The device of claim 1, wherein said microchannel is covered by one of a lamination, a sealing plate and a plate fixed over said microchannel and maintained by external pressure.

55. The device of claim 1, wherein said at least one electrode is composed of a conductive surface selected from a metal surface, carbon and a liquid/liquid interface.

56. The device of claim 1, wherein said covered microchannel contains a biological compound.

57. The device of claim 56, wherein said biological compound is selected from an enzyme, an antibody, an antigen, an oligonucleotide, DNA, a DNA strain or a cell.

58. The device of claim 56, wherein said biological compound is immobilized in said covered microchannel.

59. The device of claim 1, wherein the application of said pressure difference can be stopped.

60. The device of claim 59, wherein the stopping of the application of said pressure difference is performed by mechanically blocking one of said inlet and said outlet of said microchannel.

61. The device of claim 59, wherein the stopping of the application of said pressure difference is performed by adding a liquid immiscible with said solution to at least one of said inlet and said outlet.

62. The device of claim 1, wherein said flow of solution is used in an affinity sorbent assay in order to perform incubation of a solution in said microchannel and/or washing of said microchannel.

63. The device of claim 1, wherein said at least one integrated electrode is not in direct contact with said solution in said microchannel.

64. The device of claim 1, wherein said at least one integrated electrode is adapted to detect an analyte by amperometric measurement.

65. The device of claim 1, wherein said integrated electrode is adapted to simultaneously detect an analyte by electrochemistry and monitor the solution flow by amperometric measurement.

66. The device of claim 1, wherein the solution flow within said microchannel is continuously monitored at the precise location of said integrated electrode by amperometric measurement during all the steps of an analytical assay preceding the detection of the analyte.

67. A method of performing an analytical assay in a microfluidic system with amperometric flow monitoring, said method comprising the steps of:
(a) providing a microfluidic system comprising at least one covered microchannel having an inlet and an outlet as well as at least one electrode integrated in a wall portion of said microchannel, said electrode having a precise size and location in said microchannel;
(b) depositing a solution at the inlet of said covered microchannel;
(c) applying a pressure difference between the inlet and outlet of said microchannel in order to generate a flow of said solution in said microchannel;
(d) monitoring the solution flow at said integrated electrode by amperometric measurement; and
(e) electrochemically detecting an analyte of interest by means of said integrated electrode.

68. The method of claim 67, wherein steps b) to d) are repeated in order to perform a multi-step assay.

69. The method of claim 67, wherein said pressure difference is generated by imposing an acceleration to the microfluidic system.

70. The method of claim 69, wherein the flow of solution within said covered microchannel is generated without any pumping means.

71. The method of claim 69, wherein said acceleration is induced by the displacement of said microfluidic system or of a solid support on or in which said microfluidic system is placed.

72. The method of claim 71, wherein said displacement consists either in rotating or in vertically lifting said microfluidic system or its solid support, so as to generate a gravitation force or, respectively, a centrifugal force.

73. The method of claim 67, comprising stopping the application of said pressure difference before the electrochemical detection of said analyte of interest.

74. The method of claim 73, wherein the step of stopping the application of pressure difference comprises mechanically blocking one of said inlet and said outlet of said microchannel.

75. The method of claim 73, wherein the step of stopping the application of pressure difference comprises adding a liquid immiscible with said solution to at least one of said inlet and said outlet.

76. The method of claim 67, wherein an analyte detected in the assay is directly used to monitor said solution flow by measuring an electrochemical property of said solution comprising said analyte.

77. The method of claim 67, wherein an analyte is detected by amperometry at said at least one electrode.

78. The method of claim 77, wherein the monitoring of the solution flow and the detection of an analyte is performed simultaneously by amperometry at said integrated electrode.

79. The method of claim 67, wherein the solution flow is continuously monitored during a multi-step assay, except during the electrochemical detection of said analyte of interest.

80. The method of claim 67, wherein the solution flow is continuously monitored during a multi-step assay, except during the electrochemical detection of said analyte of interest.

81. The method of claim 67, for performing chemical and/or biological analysis with electrochemical detection.

82. The method of claim 81, for performing affinity assays such as immunological, oligonucleotide, hybridization or protein interaction assays.
